(19) United States
(12) Patent Application Publication
(10) Pub. No.: US 2026/0137482 A1
(43) Pub. Date: May 21, 2026

(54) METHODS AND APPARATUS FOR STABILIZATION OF SURGICAL ROBOTIC ARMS

(71) Applicant: LEM Surgical AG, Bern (CH)

(72) Inventors: Yossi BAR, Muri bei Bern (CH); Antonio BOZURIC, Koniz (CH)

(73) Assignee: LEM Surgical AG, Bern (CH)

(21) Appl. No.: 19/452,057

(22) Filed: Jan. 16, 2026

Related U.S. Application Data
(63) Continuation of application No. PCT/EP2024/072673, filed on Aug. 9, 2024.
(60) Provisional application No. 63/532,753, filed on Aug. 15, 2023.

(51) Int. Cl. A61B 90/50; A61B 34/30; B25J 9/16

(52) U.S. Cl. CPC A61B 90/50; A61B 34/30; B25J 9/1682; A61B 2090/508

(57) ABSTRACT

A stabilized surgical robotic system includes a surgical robotic chassis having at least first and second surgical robotic arm assemblies mounted thereon. A stabilization member having first and second attachment regions is detachably coupled to the first and second surgical robotic arm assemblies, respectively, while at least the first surgical robotic arm assembly is left free to hold and manipulate a surgical tool. A surgical robotic controller repositions at least one of the first and second surgical robotic arm assemblies while said assemblies are coupled by the stabilization member.

### CROSS REFERENCE

This application is a continuation of PCT No. PCT/EP2024/072673, filed Aug. 9, 2024, which claims the benefit of U.S. Provisional Application No. 63/532,753, filed Aug. 15, 2023, which application is incorporated herein by reference in its entirety.

### BACKGROUND

### Field

The disclosed technology relates generally to medical apparatus and methods and, more particularly, to surgical robotic systems and methods for stabilizing surgical robotic arms during procedures using such surgical robotic system.

Robotic surgery and surgical robots are now in common use. A typical robotic surgical procedure uses multiple surgical tools deployed on two, three, or more surgical robotic arms under the control of a surgical robotic controller. The surgical robotic arms and the tools they carry are typically navigated using robotically controlled cameras and/or other sensors, often in combination with kinematic tracking and placement of the surgical robotic arms and surgical tools.

Certain robotic surgical procedures require that the robotic arms apply large forces to the patient anatomy. For example, drilling a vertebra and placing a pedicle screw requires exerts significant torque on the vertebra, resulting in counter forces that can displace the drills and screwdrivers being used. Even a small displacement can deviate the intended trajectory of the drill and/or pedicle screw which can materially reduce the quality of the screw placement.

To address these concerns, surgical robots which are intended for use in spinal and other orthopedic procedure which involve drilling, sawing, and grinding bone will utilize heavy-duty robot arms which can resist significant counter forces. It has been found, however, that the counter forces encountered in some spinal and other robotic surgical procedures can exceed the capability of even heavy-duty robotic arms to hold a stable trajectory by themselves.

There is thus a need for apparatus and methods that can be used with surgical robotic systems to stabilize the surgical robotic arms of those systems when being used in spinal and other heavy duty surgical procedures. Such systems and methods should require minimum or no modification of the surgical robotic system itself and should instead allow existing components of the surgical robotic systems, such as a first surgical robotic arm, to be used to stabilize a second surgical robotic arm that is being used to perform a heavy-duty operation. While being particularly useful in performing spinal and other heavy duty surgical procedures, these apparatus and methods should be useful with many surgical robotic systems, even those which are not intended for spinal and other heavy duty surgical procedures. At least some of these objectives will be met by the technologies disclosed herein.

### Background Art

Articulated arms for holding surgical tools during surgical procedures are described in U.S. Pat. Nos. 4,431,329; 3,910,538 and D644085, assigned to Baitella AG and are commercially available under the Fisso tradename. Commonly owned publications and applications describing surgical robots and tools include PCT application nos. PCT/IB2022/052297 (published as WO2022/195460); PCT/IB2022/058986 (published as WO2023/067415); PCT/IB2022/058972 (published as WO2023/118984); PCT/IB2022/058982 (published as WO2023/118985); PCT/IB2022/058978 (published as WO2023/144602); PCT/IB2022/058980 (published as WO2023/152561); PCT/IB2023/055047 (published as WO2023/223215); PCT/IB2022/058988 (published as WO2023/237922); PCT/IB2023/055439; PCT/IB2023/055662; PCT/EP2024/052338; PCT/IB2023/055663; PCT/EP2024/052338; PCT/IB2023/056911; PCT/EP2024/052353; PCT/EP2024/068766; and U.S. provisional application Nos. 63/532,753, 63/568,102, 63/578,395; 63/606,001; 63/609,490; 63/615,076; 63/634,161, the full disclosures of each of which are incorporated herein by reference in their entirety.

### SUMMARY

The disclosed technologies provide systems and methods for stabilizing surgical robotic arms used in surgical robotic systems. In particular, the disclosed technology provides systems and methods for stabilizing robotic arms used in robotic surgical systems where the surgical robotic arms may be exposed to high counter forces, such as in spinal and other orthopedic robotic surgeries where a surgical robotic arm may be exposed to high counter forces or moments resulting from the application of high torque to the bony anatomy, such as in drilling, screwing, grinding, sawing, and the like. By stabilizing the robotic surgical arm which is holding the surgical tool performing a heavy-duty operation, unintended displacement of the surgical tool performing the operation can be minimized or eliminated, to maintain a desired tool trajectory. For example, the very fine tolerances that are required in drilling bone for pedicle screws and other purposes can be maintained by stabilizing the robotic surgical arm holding the surgical drill even when that arm is subjected to significant counter forces.

Accordingly, the disclosed technologies provide systems and methods for stabilizing surgical robotic arms by deploying a stabilization member, such as a stabilization member, which is attachable to one or more surgical robotic arms in a surgical robotic system. In some embodiments, a first robotic surgical arm may hold the stabilization member to stabilize a second surgical robotic arm to enable a stable trajectory for a tool held by the second arm. In other embodiments, the stabilization member may stabilize two robotic arms, enabling each arm to maintain a stable trajectory for tools held by those arms. In still other embodiments, a first surgical robotic arm can stabilize a second surgical robotic arm directly without use of a stabilizing member.

The stabilization members of the disclosed technology can typically have at least one degree of freedom and be adjustable at that at least one degree of freedom in order to be able to set, and then hold, desired trajectories. In many embodiments, the stabilization members can be pivotally attached to one or two surgical robotic arms, and the stabilization members may be sterilizable so that that can be reused after deployment in the surgical field.

In a first aspect, the disclosed technology provides a stabilized surgical robotic system comprising a surgical robotic chassis having at least first and second surgical robotic arm assemblies mounted thereon. A stabilization member having first and second attachment regions can be configured to be detachably coupled to the first and second surgical robotic arm assemblies, respectively, while at least the first surgical robotic arm assembly remains free to hold and manipulate a surgical tool. A surgical robotic controller can be configured to reposition at least one of the first and second surgical robotic arm assemblies while said assemblies are coupled by the stabilization member.

In some instances, the stabilization member is pivotally attached to only one of the first and second surgical robotic arm assemblies, while in other instances, the stabilization member is pivotally attached to both the first and second surgical robotic arm assemblies. Pivotal attachment may be accomplished in a variety of ways, such as using a ball-and-socket joint, a universal joint, or any similar mechanical attachment structure.

In some instances, the stabilization member may comprise a pivotal joint disposed in at least one of the first and second attachment regions. In other instances, the stabilization member may comprise an elongate structure, for example a rigid straight shaft, configured to be pivotally attached at one end to the first surgical robotic arm assembly and to be held in a tool holder at a second end, where the tool holder is held and manipulated by the second surgical robotic arm assembly.

In some instances, the stabilization member may comprise at least one lockable articulating and/or sliding joint along its length.

In some instances, each of the first and second surgical robotic arm assemblies may comprise multiple links connected by joints and may include a base link mounted on the chassis and a distal link, where at least one of the first and second attachment regions of the stabilization member is configured to couple to the distal link of one of the surgical robotic arm assemblies.

In some instances, each of the first and second surgical robotic arm assemblies may comprise multiple links connected by joints and includes a base link mounted on the chassis and a distal link, where at least one of the first and second attachment regions of the stabilization member is configured to be held by a tool holder coupled to the distal link of one of the surgical robotic arm assemblies.

In such instances, the surgical robotic controller may be further configured to manipulate the tool holder to reposition the stabilization member while repositioning the at least one of the first and second surgical robotic arm assemblies. For example, the surgical robotic controller may be further configured to manipulate the tool holder to tighten and loosen a grip on the stabilization member to as the first and second surgical robotic arm assemblies are being repositioned.

In some instances, at least one of the first and second surgical robotic arm assembly may include an attachment feature which is configured to be detachably connected to the attachment region of said stabilization member. The attachment feature may be located on a distal link of the at least one of the first and second surgical robotic arm assembly, for example comprising a threaded receptacle formed on an outer surface of the distal link.

In some instances, the surgical robotic chassis may comprise essentially of a single rigid frame which defines a surgical robotic coordinate space. Alternatively, the surgical robotic chassis may comprise two or more rigid frames which can be rigidly interconnected to define a surgical robotic coordinate space.

In specific instances, the surgical robotic controller may be configured to kinematically reposition at least one of the first and second surgical robotic arm in the surgical robotic coordinate space. Alternatively or additionally, the surgical robotic controller may be configured to optically position and manipulate either or both the first and second surgical robotic arm assemblies.

In some instances, the surgical robotic chassis may comprise a mobile cart. In other instances, the surgical robotic chassis may comprise a surgical table.

In a second aspect, the disclosed technology provides a method for performing a surgical robotic procedure. The method comprises providing a surgical robotic chassis having at least first and second surgical robotic arm assemblies mounted thereon. First and second attachment region of a stabilization member are coupled to the first and second surgical robotic arm assemblies, respectively, and a surgical operation is performed on a patient with a tool attached to the first surgical robotic arm assembly while the first surgical robotic arm assembly remains coupled to and stabilized by the second surgical robotic arm assembly.

In some instances, coupling the first attachment region of the stabilization member to the first surgical robotic arm assembly may comprise pivotally attaching the first attachment region to a distal link of the first surgical robotic arm assembly.

In some instances, coupling the second attachment region of the stabilization member to the second surgical robotic arm assembly may comprise pivotally attaching the second attachment region to a distal link of the second surgical robotic arm assembly.

In some instances, the stabilization member comprises at least one lockable articulating joint and/or sliding joint along its length to allow the first and/or second surgical robotic arm assemblies to be repositioned while the joints are unlocked. In such instances, the surgical operation may be performed while the at least one lockable articulating joint and/or sliding joint is locked.

In some instances, coupling the second attachment region of the stabilization member to the second surgical robotic arm assembly comprises holding said second attachment region in a tool holder mounted on a distal end of the second surgical robotic arm assembly. In such instance, the methods may further comprise manipulating the second surgical robotic arm assembly and the tool holder to stabilize the first surgical robotic arm assembly and while performing the surgical operation on the patient with the tool attached to the first surgical robotic arm.

The surgical robotic system used with the disclosed technologies may be single-arm surgical robotic systems, but more often will comprise multi-arm surgical robotic systems. The disclosed technologies may be used with any known multi-arm surgical robotic system, such as teleoperated systems where multiple arms are deployed from a single origin point or teleoperated systems where multiple arms are deployed each on their own cart or chassis. In some embodiments, solely by way of example, the disclosed technologies may be used with multi-arm robotically controlled surgical robotic systems wherein multiple arms each have their own point of origin (allowing kinematic positioning control by the surgical robotic controller) but are all based on a single mobile chassis.

The disclosed technologies provide systems and methods for stabilizing surgical robotic arms in robotic surgical systems when the robotic arms are exposed to high forces and/or moments in relevant surgical applications. Most particularly, in some surgical applications, for example in spinal surgery, a surgical robotic system may be exposed to high forces or high moments in the form of high torque or excessive force feedback. These high forces may be encountered in drilling and or bone cutting applications in robotic spinal and/or orthopedic surgery, for example, undesirable feedback or skidding may occur upon first application of a drill to bone, or high torque may be encountered during drilling.

The disclosed technologies may achieve stable trajectories for surgical tasks, for example, drilling or cutting, where the required tolerances in the particular surgical applications may exceed the ability of conventional robotic arms to hold a steady trajectory in view of the forces being applied or experienced. Even such heavy-duty robotic arms may experience drift or deviation from desired coordinates when high counter forces are experienced. Most particularly, the disclosed technologies provide systems and methods for stabilization of robotic arms in a multi-arm surgical robotic system in situations where the robotic arms are encountering high forces and/or have a need to maintain stable trajectories beyond the performance limits of the robotic arms themselves.

Accordingly, the disclosed technologies provide systems and methods for stabilizing robotic arms that incorporate a stabilization member attached to one or more robotic arms in a surgical robotic system. In some embodiments, the stabilization member may be providing stabilization to one robotic arm to enable a stable trajectory, and in other embodiments, the stabilization member may provide stabilization to two robotic arms, enabling each to maintain a stable trajectory. In embodiments where the stabilization member is providing stabilization to two robotic arms, the two robotic arms may be based on single chassis, such as a mobile cart, or on an integrated chassis comprising two or more rigidly joined components, as described in commonly owned PCT application no. PCT/EP2024/052353, filed on Jan. 31, 2024, the full disclosure of which is incorporated herein by reference in its entirety. A single chassis may be mobile and may be configured to be selectively placed under, and removed from, a surgical bed during a robotic surgical procedure. The single chassis may also incorporate a central controller to coordinate the movement of the robotic arms based on the single chassis. Such robotic surgical system are described in commonly owned U.S. application Ser. No. 18/217,595 (published as US2023/0380916), filed on Nov. 30, 2023, the full disclosure of which is incorporated herein by reference.

In other instances, stabilization can be achieved without use of a separate stabilization member where a first surgical robotic arm can stabilize a second surgical robotic arm directly by holding a portion of a second arm with a grasping tool or other end effector held by the first arm.

The stabilization members of the disclosed technologies will typically have at least one degree of freedom and will be adjustable in that one degree of freedom in order to be able to set, and then hold, desired surgical trajectories of the robotic arms being stabilized. The adjustment means may be an articulating adjustment, such as a screw, but may also be any other adjustment means achieving the same result. In particular, the stabilization member disclosed herein should allow for a wide range of positions of the two robotic arms being stabilized through positioning of those arms by the surgical robotic system or the operator and then adjustment of the stabilization member to keep the robotic arms in the desired positions. One of skill in the art will understand that at least one degree of freedom for the stabilization member is required for this task, but that more degrees of freedom may be possible or even desirable.

The stabilization members of the disclosed technologies are configured to be deployed in the surgical field and accordingly should be amenable to sterilization. The stabilization members may be constructed of any material that is surgically acceptable and that can be sterilized by known sterilization methods. Solely by way of example, the stabilization member may be constructed of medical grade stainless steel or titanium.

The robotic arms being stabilized in the disclosed systems and methods may incorporate end effectors. The end effectors may be grippers, tool holders, or other conventional end effectors. The end effectors may be configured to hold surgical tools in desirable surgical trajectories.

In one representative embodiment and associated method, the stabilization member may be joined to the end effectors of the two robotic arms being stabilized by any suitable attachment method. The attachment method may incorporate a ball, screw, pin or other mechanical means, but could also be magnetic or electromagnetic. In this embodiment and associated methods, the attachment of the stabilization member to the end effector allows for full functionality of the end effectors of each of the robotic arms. For example, if the end effector is a gripper, each of the robotic arms can still hold a tool in their associated end effector/gripper and, thus, maintain a desired surgical trajectory for the tools with the stabilization member in place.

In another representative embodiment and associated method, the stabilization member may be joined to the end effectors of the two robotic arms being stabilized by any suitable attachment method or may be grasped by the end effectors. In this embodiment and associated methods, the attachment of the stabilization member to the end effector, or the grasping of the stabilization member by the end effector, allows for full functionality of one of the end effectors of the robotic arms but blocks the functionality of the other end effector (in terms of being able to maintain and operate a surgical trajectory). For example, this may happen if one end of the stabilization member is held by the gripper of one of the robotic arms and the other end of the stabilization member is merely attached to the second end effector by any suitable attachment means. In this example, if the end effector is a gripper, one of the robotic arms has lost its gripper functionality since the gripper is grasping one end of the stabilization member and one gripper is still able to hold a surgical tool, resulting in a desired surgical trajectory being maintained for that tool. This may be desirable in situations where only one surgical trajectory is required or where even more force stabilization is warranted.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a side view of a surgical robot having first and second surgical robotic arms which carry bone grinding tools a third surgical robotic arm carrying a navigation camera, in accordance with some embodiments.

FIG. 2 is a side view of a first exemplary stabilizing member of the disclosed technology suitable for use with the surgical robot of FIG. 1, in accordance with some embodiments.

FIG. 2A is a detailed view of a pivoting connector of the stabilizing member taken along line 2A-2A of FIG. 2, in accordance with some embodiments.

FIG. 3 is an enlarged sideview of the stabilizing member of FIG. 2 mounted on the surgical robot of FIG. 1, in accordance with some embodiments.

FIG. 4 is a side view of a second exemplary stabilizing member of the disclosed technology suitable for use with the surgical robot of FIG. 1 shown in a tool holder with portions broken away, in accordance with some embodiments.

FIG. 5 is an enlarged sideview of the stabilizing member of FIG. 4 mounted on the surgical robot of FIG. 1, in accordance with some embodiments.

FIG. 6 is an enlarged sideview of the surgical robot of FIG. 1 showing a terminal link of a first surgical robotic arm directly connected to a terminal link of a second surgical robotic arm which is performing a grinding operation on patient bone, in accordance with some embodiments.

### DETAILED DESCRIPTION

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

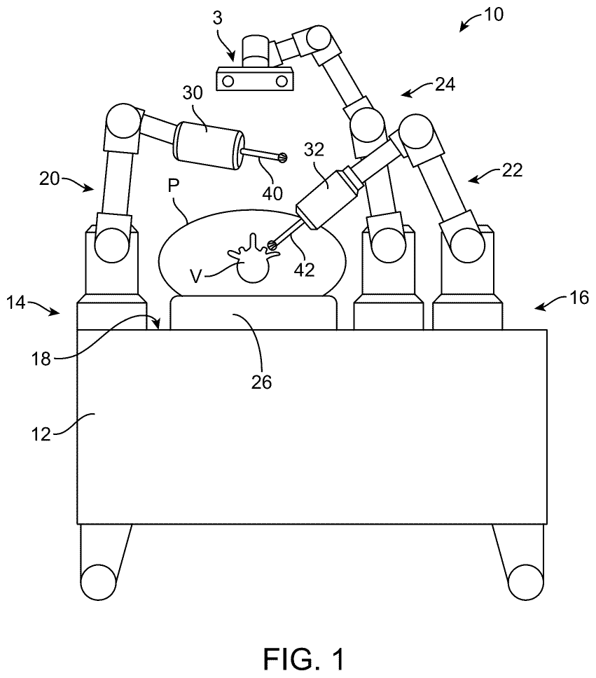

An exemplary robotic surgical system 10 suitable for use with the stabilizing members and methods of the disclosed technology is shown in FIG. 1. The robotic surgical system 10 may comprise a chassis 12, typically a single, rigid frame, which provides a base or platform for three robotic arms 20, 22 and 24 that are placed relatively far apart on opposite longitudinal ends 14 and 16 of an upper surface 18 of the chassis 12, typically approximately one meter apart, thus allowing for desirable attributes such as reachability, maneuverability, and an ability to apply significant force. In the illustrated embodiment, robotic surgical arm 20 is on the first end 14 of the chassis 12 and robotic surgical arms 22 and 24 are on the second end 16 of the chassis. The chassis is often but not necessarily a mobile, e.g., being in the form of a mobile cart as described in commonly owned PCT application nos. PCT/IB2022/052297 (published as WO2022/195460), previously incorporated herein by reference. In other embodiments and implementations, the surgical arms 20, 22 and 24 can be mounted on a base or other structure of a surgical table. Placement of the robotic surgical arms on a common, stable platform allows the arms to be moved kinematically or otherwise within a common robotic coordinate system having a single origin point under the control of a surgical robotic controller, typically an on-board controller (not shown) having a user interface, such as display screen. Alternatively, the controller can be located at a remote workstation.

The single, rigid chassis of the disclosed technology can usually comprise, consist of, or consist essentially of a single mobile cart, as disclosed for example in commonly owned PCT application nos. PCT/IB2022/052297 (published as WO2022/195460), the full disclosure of which has been previously incorporated herein by reference. In other instances, however, the single, rigid chassis may comprise separate modules, platforms, or components, that are assembled at or near the surgical table, as described for example in commonly owned PCT Application PCT/EP2024/052353, entitled Integrated Multi-Arm Mobile Surgical Robotic System, filed on Jan. 29, 2024, the full disclosure of which is incorporated herein by reference. The only requirement of the single, rigid chassis is that it provide a stable base for all the surgical arms so that they may be accurately and precisely kinematically positioned and tracked by the surgical robotic controller in a single surgical robotic coordinate space.

The chassis 12 of the robotic surgical system 10 is optionally configured to be temporarily placed under a surgical table 26 when performing the robotic surgical procedure, allowing the robotic surgical system 10 to be stored remotely before and after the procedure. The robotic arms 20, 22, and 24 may optionally be configured to be retracted into the chassis 12 of the robotic surgical system, allowing the system to be moved into or out of the surgical field in a compact configuration.

The robotic surgical arms 20, 22 and 24 are typically “multilink” structures with arms 20 and 22 having terminal links 30 and 32 which are configured to hold tools, tool holders, flanges, end effectors, or the like, depending on the specific construction of the robot arm. For simplicity of explanation, FIG. 1 illustrates these components as terminal links 30 and 32 of the robotic arms 20 and 22 which directly hold grinding tools 40 and 42. In other embodiments, not illustrated, grinders or other surgical tools could be held by tool holders as described, for example, in commonly owned PCT Application PCT/EP2024/068766, filed on Jul. 4, 2024, the full disclosure of which has been previously incorporated herein by reference. In place of or in addition to the tool holders, flanges (not illustrated could be mounted at the distal links of either or both robotic surgical arms 20 and 22. Such flanges could contain electronics and other sensitive system components that cannot be sterilized under harsh condition. The third surgical robotic arm 24 will typically carry a navigation camera 34 or other sensor for monitoring the robotic surgical space.

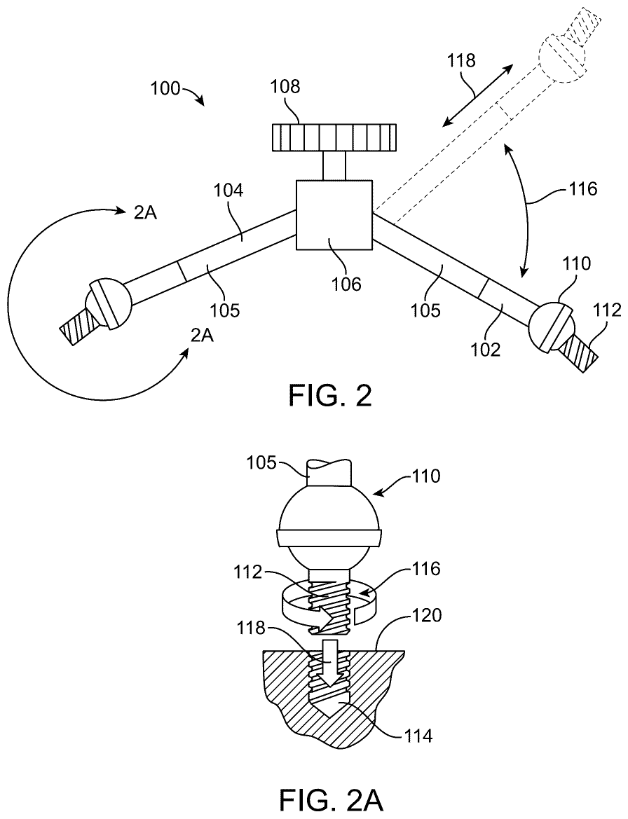

Referring now to FIGS. 2 and 2A, a first exemplary stabilizing member 100 includes first and second locking arms 102 and 104 each comprising a shaft 105. As illustrated, the shafts 105 are elongate, linear elements each having a proximal end attached to a hub 106 and a distal end terminating in a pivotal connector 110. Curved and other non-linear shaft designs could also be employed if circumstances warrant such designs. The proximal shaft ends are typically mounted in the hub 106 to allow pivotal rotation, as shown in broken line and indicated by arrow 116, as well as translation in and out of the hub, as shown by arrow 118. In this way, the distances and angulations between of each of the pivotal connectors 112 can be widely adjusted. A locking knob 108 may be provided to allow the arm positions to be released for adjustment and locked when in a desired configuration. The details of such locking mechanisms are well known in the art and described, for example, U.S. Pat. Nos. 4,431,329; 3,910,538, the full disclosures of which are incorporated herein by reference.

As shown in FIG. 2A, the pivotal connectors 110 may comprise a ball-and-socket joint or other well-known mechanism, such as a universal joint, which allow a threaded shaft 112 to freely pivot relative to an axis of the shaft 105. The threaded shaft 112 may be attached to the target surgical arm link 30 or 32 by screwing the shaft into a mating receptacle 118 on an outer surface 120 of the link or by any one of many other well-known fastening and connecting techniques.

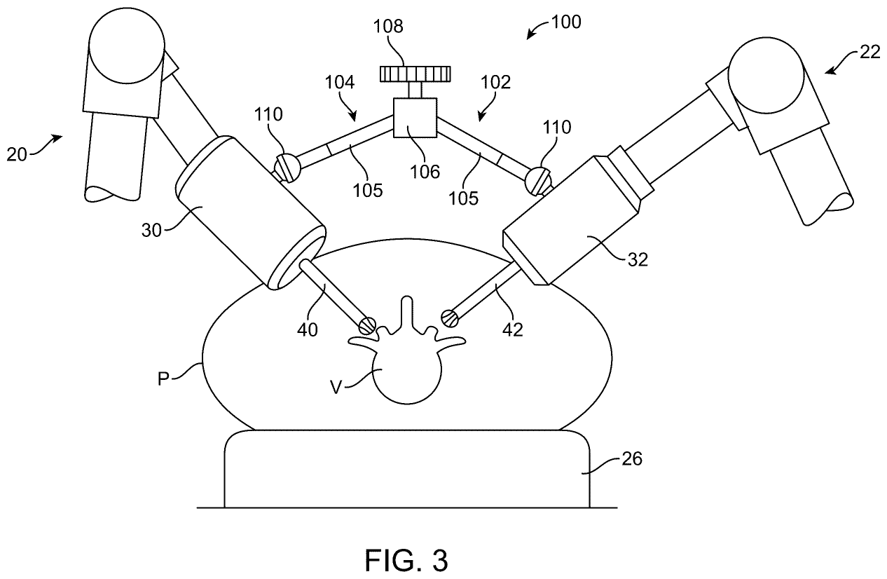

Referring now to FIG. 3, the stabilizing member 100 may be used to couple links 30 and 32 of the robotic surgical system 100 while the surgical robotic arms 20 and 22 are each manipulating a tool to perform an operation on patient P. More specifically, the robotic controller positions the surgical robotic arms 20 and 22 to place grinding tools 40 and 42 proximate target locations on a vertebra V of the patients spine. The positioning could be automatically performed by the controller or be under the direct control of the patient. Once the grinding tools 40 and 42 are properly positioned, the pivotal connectors 110 of the stabilizing member 100 can be secured to each of the terminal links 30 and 32, typically as shown in FIG. 2A. The positions of the surgical robotic arms 20 and 22 can then be adjusted and, after the locations have been finalized, the hub 106 can be locked to rigidly interconnect the terminal links 30 and 32. The grinders 40 and 42 can then be used to perform grinding on the vertebra V while each of the arms 20 and 22 of the stabilizing member 100 will support and stabilize the position of the other arm during the grinding operations. In some embodiments, the hub 106 can be released and the locking arm 102 and 104 be repositioned one or more times during any one operation. While the surgical robotic arms 20 and 22 will not normally be repositioned during the grinding operation itself, the grinders 40 and 42 can be movable relative to the stabilized links 30 and 32 using tool holders, end effectors, or the like.

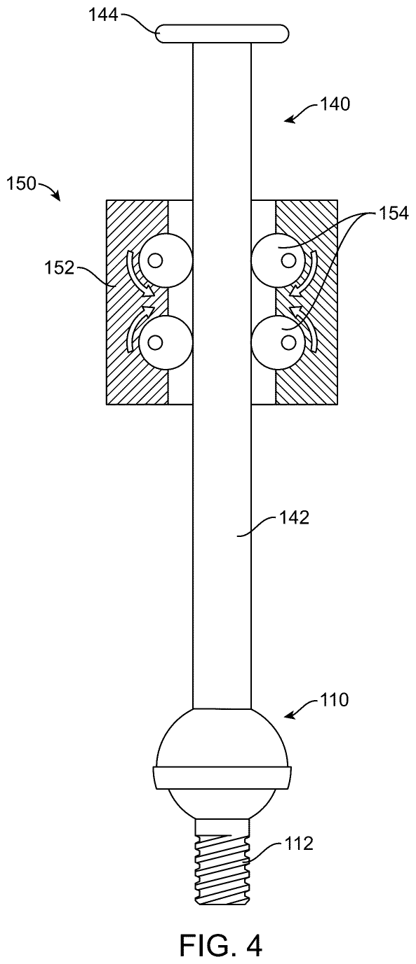

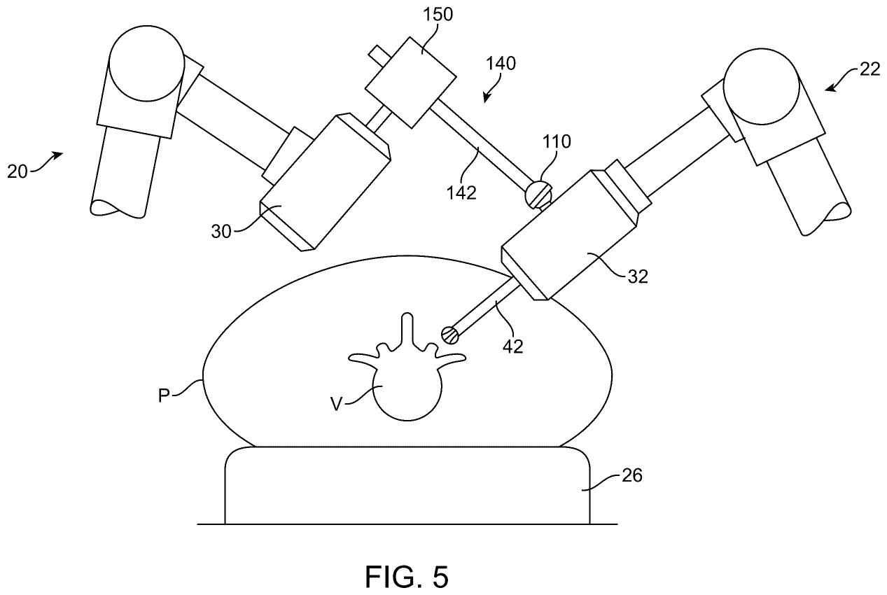

A second stabilizing member 140 is illustrated in FIG. 4. The stabilizing member 140 may comprise a single elongate shaft 142, typically a rigid cylindrical rod or other member, having a connector 110 (which can be the same as that illustrated in FIGS. 2 and 2A). The elongate shaft 142 can be linear with a straight axis but could have curved or other non-linear designs should circumstances warrant it. Unlike stabilizing member 100 which is intended to be passively interconnected between two robotic arm links, stabilizing member 140 is intended to be held in a tool holder 150 which is attached to the distal link 30 of the first surgical robot arm 20, as shown in FIG. 5. Suitable tool holders 150 are described, for example, in commonly owned PCT Application no. PCT/EP2024/068766, the full disclosure of which has been previously incorporated herein by reference and includes four gripper rollers 154 in a housing 152. Placement of the stabilizing member 140 in tool holder 150 allows the first surgical robotic arm 20 to actively reposition the stabilizing member 140 as the grinding or other surgical operation is being performed by the second surgical robotic arm 22. That is, as the second surgical robotic arm 22 is repositioned to continue the grinding or other surgical procedure, the first surgical robotic arm 20 can be automatically repositioned by the robotic controller (typically kinematically) to track the second arm movements to maintain a consistent stabilizing force.

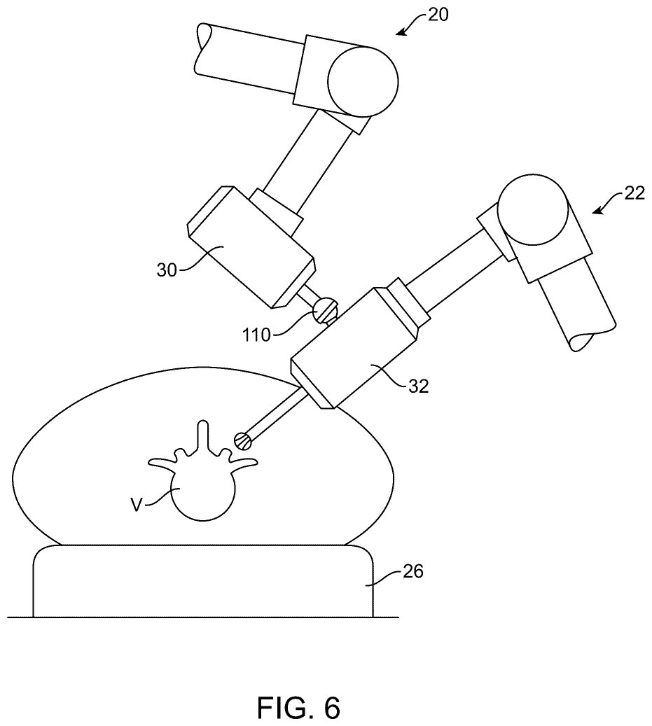

Referring now to FIG. 6, in some instances, the disclosed methods can be performed without the use of a stabilizing member as described previously. In such instances, the terminal link 30 of the first surgical robotic arm 20 can be attached directly to the terminal link 32 of the second surgical robotic arm. While a pivotal connector 110 may still be used as illustrated, in other instances the terminal link 30 of the first surgical robot arm 30 could carry a large, specialized gripper configured to releasably grip the exterior of the terminal link 32 of the second surgical robotic arm 22 (not illustrated). Other direct attachment modes can also be used, including magnetic attachment, interlocking brackets, and the like.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

List of Reference Numbers.

| | |
|---|---|
| 10 | Robotic surgical system |
| 12 | Chassis |
| 14 | First end |
| 16 | Second end |
| 18 | Upper surface |
| 20 | Robotic surgical arm (first) |
| 22 | Robotic surgical arm (second) |
| 24 | Robotic surgical arm (third) |
| 26 | Surgical table |
| 30 | Terminal link |
| 32 | Terminal link |
| 34 | Navigation camera |
| 40 | Grinding tool |
| 42 | Grinding tool |
| 100 | Stabilizing member |
| 102 | Locking arm |
| 104 | Locking arm |
| 105 | Shaft |
| 106 | Hub |
| 108 | Knob |
| 110 | Pivotal connector |
| 112 | Threaded shaft |
| 114 | Threaded receptacle |
| 116 | Arrow |
| 118 | Arrow |
| 140 | Stabilizing member |
| 142 | Shaft |
| 144 | Cap |
| 150 | Tool holder |
| 152 | Housing |
| 154 | Gripper rollers |

What is claimed is:

1. A stabilized surgical robotic system comprising:
a surgical robotic chassis;
at least first and second surgical robotic arm assemblies mounted on the chassis;
a stabilization member having first and second attachment regions configured to be detachably coupled to the first and second surgical robotic arm assemblies, respectively, while leaving at least the first surgical robotic arm assembly free to hold and manipulate a surgical tool; and
a surgical robotic controller configured to reposition at least one of the first and second surgical robotic arm assemblies while said assemblies are coupled by the stabilization member.

2. The stabilized surgical robotic system of claim 1, wherein the stabilization member is pivotally attached to at least one of the first and second surgical robotic arm assemblies.

3. The stabilized surgical robotic system of claim 1, wherein the stabilization member is pivotally attached to at least one of the first and second surgical robotic arm assemblies.

4. The stabilized surgical robotic system of claim 2, wherein the stabilization member comprises a pivotal joint disposed in at least one of the first and second attachment regions.

5. The stabilized surgical robotic system of claim 1, wherein the stabilization member comprises at least one lockable articulating joint and/or sliding joint along its length.

6. The stabilized surgical robotic system of claim 1, wherein each of the first and second surgical robotic arm assemblies comprises multiple links connected by joints and includes a base link mounted on the chassis and a distal link, and wherein at least one of the first and second attachment regions of the stabilization member is coupled to the distal link of one of the surgical robotic arm assemblies.

7. The stabilized surgical robotic system of claim 1, wherein each of the first and second surgical robotic arm assemblies comprises multiple links connected by joints and includes a base link mounted on the chassis and a distal link, and wherein at least one of the first and second attachment regions of the stabilization member is configured to be held by a tool holder coupled to the distal link of one of the surgical robotic arm assemblies.

8. The stabilized surgical robotic system of claim 7, wherein the surgical robotic controller is further configured to manipulate the tool holder to reposition the stabilization member while repositioning the at least one of the first and second surgical robotic arm assemblies.

9. The stabilized surgical robotic system of claim 7, wherein the surgical robotic controller is further configured to manipulate the tool holder to tighten and loosen a grip on the stabilization member to as the first and second surgical robotic arm assemblies are being repositioned.

10. The stabilized surgical robotic system of claim 1, wherein at least one of the first and second surgical robotic arm assembly includes an attachment feature which is configured to be detachably connected to the attachment region of said stabilization member.

11. The stabilized surgical robotic system of claim 10, wherein the attachment feature is located on a distal link of the at least one of the first and second surgical robotic arm assembly.

12. The stabilized surgical robotic system of claim 10, wherein the attachment feature comprises a threaded receptacle formed on an outer surface of the distal link.

13. The stabilized surgical robotic system of claim 1, wherein the surgical robotic chassis comprises essentially of a single rigid frame which defines a surgical robotic coordinate space.

14. The stabilized surgical robotic system of claim 1, wherein the surgical robotic chassis comprises two or more rigid frames which can be rigidly interconnected to define a surgical robotic coordinate space.

15. The stabilized surgical robotic system of claim 13, wherein the surgical robotic controller is configured to kinematically reposition at least one of the first and second surgical robotic arm in the surgical robotic coordinate space.

16. The stabilized surgical robotic system of claim 1, wherein the surgical robotic chassis comprises a mobile cart.

17. The stabilized surgical robotic system of claim 1, wherein the surgical robotic chassis comprises a surgical table.

18. A method for performing a surgical robotic procedure, said method comprising:
providing a surgical robotic chassis having at least first and second surgical robotic arm assemblies mounted thereon;
coupling a first attachment region of a stabilization member to the first surgical robotic arm assembly;
coupling a second attachment region of the stabilization member to the second surgical robotic arm assembly; and
performing a surgical operation on a patient with a tool attached to the first surgical robotic arm assembly while the first surgical robotic arm assembly remains coupled to and stabilized by the second surgical robotic arm assembly.

19. The method of claim 18, wherein coupling the first attachment region of the stabilization member to the first surgical robotic arm assembly comprises pivotally attaching the first attachment region to a distal link of the first surgical robotic arm assembly.

20. The method of claim 18, wherein coupling the second attachment region of the stabilization member to the second surgical robotic arm assembly comprises pivotally attaching the second attachment region to a distal link of the second surgical robotic arm assembly.

21. The method of claim 18, wherein the stabilization member comprises at least one lockable articulating joint and/or sliding joint along its length to allow the first and/or second surgical robotic arm assemblies to be repositioned while the joints are unlocked.

22. The method of claim 18, wherein the surgical operation is performed while the at least one lockable articulating joint and/or sliding joint is locked.

23. The method of claim 18, wherein coupling the second attachment region of the stabilization member to the second surgical robotic arm assembly comprises holding said second attachment region in a tool holder mounted on a distal end of the second surgical robotic arm assembly.

24. The method of claim 18, further comprising manipulating the second surgical robotic arm assembly and the tool holder to stabilize the first surgical robotic arm assembly and while performing the surgical operation on the patient with the tool attached to the first surgical robotic arm.

25. A method for performing a surgical robotic procedure, said method comprising:
providing a surgical robotic chassis having at least first and second surgical robotic arm assemblies mounted thereon;
coupling an attachment region of the first surgical robotic arm assembly to an attachment region of the second surgical robotic arm assembly; and
performing a surgical operation on a patient with a tool attached to the first surgical robotic arm assembly while the first surgical robotic arm assembly remains coupled to and stabilized by the second surgical robotic arm assembly.

26. The method of claim 25, wherein coupling the attachment region of the first surgical robotic arm assembly to the attachment region of the second surgical robotic arm assembly comprises coupling a terminal link of the first surgical robotic arm assembly to a terminal link of the second surgical robotic arm assembly.

27. The method of claim 25, wherein coupling the terminal link of the first surgical robotic arm assembly to the terminal link of the second surgical robotic arm assembly comprises pivotally coupling the terminal link of the first surgical robotic arm assembly to the terminal link of the second surgical robotic arm assembly.

28. The method of claim 25, wherein coupling the attachment region of the first surgical robotic arm assembly to the attachment region of the second surgical robotic arm assembly comprises grasping a terminal link of the first surgical robotic arm assembly with a grasper disposed on the second surgical robotic arm assembly.

29. The method of claim 25, further comprising manipulating the second surgical robotic arm assembly and the tool holder to stabilize the first surgical robotic arm assembly and while performing the surgical operation on the patient with the tool attached to the first surgical robotic arm.

30. A robotic arm stabilization system comprising:
a robotic system comprising at least two robotic arms; and
a stabilization member connected to the at least two robotic arms at connection points at or near end effectors of each of the robotic arms;
whereby the connection of the stabilization member to the at least two robotic arms allows at least one of the at least two robotic arms to maintain a desirable operating trajectory.

31. The robotic arm stabilization system of claim 30, wherein the end effectors are grippers capable of holding tools.

32. The robotic arm stabilization system of claim 31, wherein the attachment of the stabilization member to the grippers of the at least two robotic arms allows the grippers to maintain their ability to hold tools.

33. The robotic arm stabilization system of claim 32, wherein allowing the grippers to maintain their ability to hold tools allows for the maintenance of each of a surgical trajectory of each of the at least two robotic arms.

34. The robotic arm stabilization system of claim 33, wherein the attachment of the stabilization member to the grippers of the at least two robotic arms only allows one of the grippers to maintain its ability to hold a tool.

35. The robotic arm stabilization system of claim 34, wherein only allowing one of the grippers to maintain its ability to hold a tool allows for the maintenance of the surgical trajectory of the robotic arm holding the end effector that has maintained its ability to hold a tool.

36. The robotic arm stabilization system of claim 30, wherein the stabilization member is adjustable along at least one degree of freedom to allow for freedom of positioning of the at least two robotic arms in desirable positions in a surgical field.

37. The robotic arm stabilization system of claim 30, wherein the stabilization member is sterilizable.

38. The robotic arm stabilization system of claim 30, wherein the at least two robotic arms are based on a single chassis.

39. The robotic arm stabilization system of claim 38, wherein the single chassis is mobile and can be selectively positioned under, and removed from under, a surgical table before, during or after a surgical procedure.
